(19) United States
(12) Patent Application Publication
(10) Pub. No.: US 2021/0098121 A1
(43) Pub. Date: Apr. 1, 2021

(54) SYSTEMS AND METHOD FOR ANALYZING OPERATIONAL RHYTHMS IN HOSPITAL TREATMENT

(71) Applicant: Children's Hospital Medical Center, Cincinnati, OH (US)

(72) Inventors: Marc D. Ruben, Cincinnati, OH (US); John B. Hogenesch, Cincinnati, OH (US); David F. Smith, Cincinnati, OH (US)

(21) Appl. No.: 17/038,908

(22) Filed: Sep. 30, 2020

Related U.S. Application Data
(60) Provisional application No. 62/908,274, filed on Sep. 30, 2019.

(51) Int. Cl. G16H 40/20; G16H 50/70; G16H 70/20; G16H 20/40; G16H 20/70; G16H 20/10; G16H 50/20; G16H 10/60; A61B 5/00

(52) U.S. Cl. CPC G16H 40/20; G16H 50/70; G16H 70/20; G16H 20/40; A61B 5/4848; G16H 20/10; G16H 50/20; G16H 10/60; G16H 20/70

(57) ABSTRACT

The present disclosure relates to analyzing operational rhythms in hospital treatment and providing recommendations for hospital treatment based on the same. In an exemplary embodiment, a server comprises one or more processors; and memory comprising instructions that, when executed, cause the one or more processors to receive, from a database, treatment data corresponding to a treatment provided to a plurality of patients. The memory further comprises instructions that, when executed, cause the one or more processor to determine a preferred treatment regimen based upon the treatment data, wherein the preferred treatment regimen comprises a time of day the treatment is more effective than other times of the day based upon responses to the treatment. The memory also comprises instructions that, when executed, cause the one or more processor to provide a notification to a user interface to provide the treatment at the time of the day the treatment is more effective.

### CROSS-REFERENCE FOR RELATED APPLICATION

This application claims priority to U.S. Provisional Patent Application No. 62/908,274, filed on Sep. 30, 2019, the entire disclosure of which is expressly incorporated by reference herein.

### STATEMENT REGARDING FEDERALLY SPONSORED RESEARCH OR DEVELOPMENT

This invention was made with government support under NS054794 awarded by the National Institutes of Health. The government has certain rights in the invention.

### BACKGROUND AND SUMMARY OF THE DISCLOSURE

The present disclosure relates generally to analyzing operational rhythms in hospital treatment and providing recommendations for hospital treatment based on the same.

Hospitals operate 24 hours a day, and it is assumed that important clinical decisions occur continuously around the clock. However, many aspects of hospital operation occur at specific times of day, including medical team rounding and shift changes. Embodiments disclosed herein address this problem by using big-data analysis necessarily rooted in computer technology to determine whether hospital operation occurring at specific times of day impacts patient care.

In particular, the embodiments disclosed herein address this problem by analyzing the daily distribution of ˜500,000 doses of 12 separate therapeutics in 1,546 inpatients at a major children's hospital in the United States from 2010 to 2017 using techniques necessarily rooted in computer technology, i.e., using big data analysis. The embodiments track order time (i.e., when a care provider places an electronic request for a therapeutic), which day the electronic request was requested, and dosing time (i.e., when the patient receives the therapeutic). Order times were time-of-day dependent with relatively few orders occurring at night. For example, order times were marked by distinct morning-time Q:6 surges and overnight lulls. Nearly one-third of all 103,847 orders for treatment were placed between 8:00 AM and 12:00 PM. First doses from each order were also rhythmic but shifted by 2 hours. These 24-hour rhythms in orders and first doses were remarkably consistent across therapeutics, diagnosis, and hospital units. This rhythm in hospital medicine coincided with medical team rounding time, not necessarily immediate medical need. Furthermore, the embodiments show that the clinical response to hydralazine, an acute antihypertensive, is dosing time-dependent and greatest at night, when the fewest doses were administered. Using this information, the embodiments disclosed herein provide notifications and recommendations to improve patient care, thereby providing a practical application.

Exemplary embodiments include but are not limited to the following examples.

In an exemplary embodiment, a server configured to analyze operational rhythms in hospital treatment and provide recommendations based on the same, comprises: one or more processors; and memory comprising instructions that, when executed, cause the one or more processors to: receive, from a database, treatment data corresponding to a treatment provided to a plurality of patients; determine a preferred treatment regimen based upon the treatment data, wherein the preferred treatment regimen comprises a time of day the treatment is more effective than other times of the day based upon responses to the treatment; and provide a notification to a user interface to provide the treatment at the time of the day the treatment is more effective.

In an example according to the previous paragraph, wherein the treatment corresponds to administration of a therapeutic, a performance of a procedure, or a combination thereof.

In an example according to the previous paragraph, wherein the procedure is a surgery, dialysis, cognitive behavioral therapy, a diagnostic test, or a combination thereof.

In an example according to any one of the two previous paragraphs, wherein the treatment is at least one therapeutic selected from the following group of therapeutics: anti-hypertensives, anti-inflammatories, analgesics, anti-infectives, antihistamines, diuretics, vasodilators, beta-blockers, inotropes, pain therapeutics, or a combination thereof.

In an example according to any one of the previous paragraphs, wherein to determine the preferred treatment regimen, the memory comprises instructions that, when executed, cause the one or more processors to use statistical methods, machine learning, or a combination thereof to determine what time of day the response to the treatment is more effective than other times of the day.

In an example according to the previous paragraph, wherein the statistical methods comprise cosinor regression.

In an example according to any one of the five preceding paragraphs, the memory comprising instructions that, when executed, cause the one or more processors to determine a delayed release formulation that corresponds to a difference between (i) a time of day when the therapeutic is more effective than other times of the day and (ii) a standard time of day a patient takes the therapeutic when the patient is at a location other than a patient care facility.

In an example according to the previous paragraph, wherein the standard time of day is in the morning.

In an example according to the previous paragraph, wherein the standard time of day is between 5 am and 9 am.

In an example according to any one of the three previous paragraphs, wherein the standard time of day is in the evening.

In an example according to the previous paragraph, wherein the standard time of day is between 8 pm and 12 am.

In an example according to any one of the previous paragraphs, wherein the treatment regimen comprises a time of day the treatment is provided.

In an example according to any one of the previous paragraphs, wherein the notification includes an anticipated dose lag for a therapeutic.

In an example according to the previous paragraph, wherein the anticipated dose lag is based on at least one hospital parameter.

In another exemplary embodiment, a method for analyzing operational rhythms in hospital treatment providing recommendations based on the same, the method comprising: receive, from a database, treatment data corresponding to a treatment provided to a plurality of patients; determine a preferred treatment regimen based upon the treatment data, wherein the preferred treatment regimen comprises a time of day the treatment is more effective than other times of the day based upon responses to the treatment; and provide a notification to a user interface to provide the treatment at the time of the day the treatment is more effective.

In an example according to the previous paragraph, wherein the treatment corresponds to administration of a therapeutic, a performance of a procedure, or a combination thereof.

In an example according to the previous paragraph, wherein the procedure is a surgery, dialysis, cognitive behavioral therapy, a diagnostic test, or a combination thereof.

In an example according to any one of the two previous paragraphs, wherein the treatment is at least one therapeutic selected from the following group of therapeutics: anti-hypertensives, anti-inflammatories, analgesics, anti-infectives, antihistamines, diuretics, vasodilators, beta-blockers, inotropes, pain therapeutics, or a combination thereof.

In an example according to any one of the four previous paragraphs, wherein determining the preferred treatment regimen comprises using statistical methods, machine learning, or a combination thereof to determine what time of day the response to the treatment is more effective than other times of the day.

In an example according to the previous paragraph, wherein the statistical methods comprise cosinor regression.

In an example according to any one of the six previous paragraphs, further comprising determining a delayed release formulation that corresponds to a difference between (i) a time of day when the therapeutic is more effective than other times of the day and (ii) a standard time of day a patient takes the therapeutic when the patient is at a location other than a patient care facility.

In an example according to the previous paragraph, wherein the standard time of day is in the morning.

In an example according to the previous paragraph, wherein the standard time of day is between 5 am and 9 am.

In an example according to any one of the three previous paragraphs, wherein the standard time of day is in the evening.

In an example according to the previous paragraph, wherein the standard time of day is between 8 pm and 12 am.

In an example according to any one of the eleven previous paragraphs, wherein the treatment regimen comprises a time of day the treatment is provided.

In an example according to any one of the twelve previous paragraphs, wherein the notification includes an anticipated dose lag for a therapeutic.

In an example according to the previous paragraph, wherein the anticipated dose lag is based on at least one hospital parameter.

In another exemplary embodiment, a method comprises receiving treatment data corresponding to a treatment provided to a plurality of patients; analyzing the treatment data to determine a treatment regimen; comparing the treatment regimen with a preferred treatment regimen; determining the treatment regimen varies from the preferred treatment regimen by a threshold; and providing a notification to update the treatment regimen based on the treatment regimen varying from the preferred treatment by the threshold.

In an example according to the previous paragraph, wherein the treatment corresponds to administration of a therapeutic, a performance of a surgery, or the administration of the therapeutic and the performance of the surgery.

In an example according to any one of the two previous paragraphs, wherein the treatment regimen and the preferred treatment regimen include at least one selected from the group of: a time of day that a procedure was performed on a patient, a time of day that a therapeutic was administered to a patient, a day of the week that a procedure is performed on a patient, and/or a day of the week that a therapeutic was administered to a patient.

In an example according to any one of the three previous paragraphs, wherein the treatment is at least one therapeutic selected from the following group of therapeutics: anti-hypertensive drugs and anti-inflammatory drugs.

In an example according to any one of the four previous paragraphs, the method further comprises determining the preferred treatment regimen.

In an example according to the previous paragraph, wherein determining the preferred treatment regimen, the method comprises uses statistical methods, machine learning, or statistical methods and machine learning.

In an example according to any one of the two preceding paragraphs, wherein to determine the preferred treatment regimen, the method comprises determining a preferred time of administration for a therapeutic.

In an example according to the previous paragraph, the method comprises recommending a delayed release formulation of the therapeutic to deliver the therapeutic at the preferred time of administration when a time of dosing cannot be accurately controlled.

In an example according to any one of the nine previous paragraphs, the method comprises receiving a signal to update the treatment regimen in response to the notification; and updating the treatment regimen in response to the signal.

In an example according to any one of the previous paragraphs, wherein the treatment is a therapeutic comprising a plurality of different forms of the therapeutic, each form having a different time release mechanism; and wherein providing a notification to update the treatment regimen, the method comprises providing a notification to administer a form of the therapeutic based on the time release mechanism of the therapeutic.

In an example according to the previous paragraph, wherein the different time release mechanism includes immediate release, extended release, or delayed release.

In an example according to any one of the twelve previous paragraphs, wherein the notification includes an anticipated dose lag for a therapeutic.

In an example according to the previous paragraph, wherein the anticipated dose lag is based on at least one hospital parameter.

In another exemplary embodiment, a method comprises determining a preferred treatment regimen for a treatment; comparing a treatment regimen with the preferred treatment regimen; determining the treatment regimen varies from the preferred treatment regimen by a threshold; and providing a notification to modify the treatment regimen based on the treatment regimen varying from the preferred treatment by the threshold.

In an example according to the previous paragraph, wherein determining the preferred treatment regimen, the method uses statistical methods, machine learning, or statistical methods and machine learning.

In an example according to any one of the previous two paragraphs, wherein to calculate the preferred treatment regimen, the method comprises determining a preferred time of administration for a therapeutic.

In an example according to the previous paragraph, the method comprises determining a delayed release formulation of the therapeutic to deliver the therapeutic at the preferred time of administration when a time of dosing cannot be accurately controlled.

In an example according to any one of the previous four paragraphs, the method comprises receiving a signal to update the treatment regimen in response to the notification; and updating the treatment regimen in response to the signal.

In an example according to any one of the previous five paragraphs, wherein the treatment is a therapeutic that comprises a plurality of different forms of the therapeutic, each form having a different time release mechanism; and wherein providing a notification to update the treatment regimen, the method comprises providing a notification to administer a form of the therapeutic based on the time release mechanism of the therapeutic.

In an example according to the previous paragraph, wherein the different time release mechanism includes immediate release, extended release, or delayed release.

In an example according to any one of the previous seven paragraphs, the notification is based on at least one hospital parameter.

### BRIEF DESCRIPTION OF DRAWINGS

A detailed description of the drawings particularly refers to the accompanying figures, in which:

FIG. 1 illustrates a flow diagram of a method for analyzing operational rhythms in hospital treatment and providing recommendations for hospital treatment based on the same;

FIG. 2 is a block diagram of an example system for analyzing operational rhythms in hospital treatment and providing recommendations for hospital treatment based on the same;

FIGS. 3A-3C illustrate daily rhythms in the timing of hydralazine treatment;

FIG. 4 illustrates rhythms in hydralazine orders and first doses across hospital units;

FIGS. 5A-5C illustrate rhythms in treatment across drug classes coincide with hospital-wide operational activity;

FIGS. 6A-6B illustrate clinical response to hydralazine varies by time of administration;

FIG. 7 illustrates the dose lag based on the time of the order, the therapeutic ordered, and the weekday the therapeutic is ordered;

FIG. 8 illustrates the longest lag of the therapeutics illustrated in FIG. 7 are for acetaminophen and diphenhydramine;

FIG. 9 illustrates the surgery lag for an appendicitis;

FIG. 10 illustrates whether there is an association between time of surgery for an appendicitis and post-operation antibiotic use or readmission; and

FIG. 11 is a block diagram of illustrative components of a computer system for analyzing operational rhythms in hospital treatment and providing recommendations for hospital treatment based on the same.

### DETAILED DESCRIPTION OF THE DRAWINGS

As stated above, hospitals operate 24 hours a day, yearlong. Caretakers are available around the clock, and it is assumed that important clinical decisions occur continuously regardless of time of day. However, many universal aspects of hospital operation occur at particular times of day, including medical team rounding and staff shift changes. The embodiments disclosed herein use big-data analysis necessarily rooted in computer technology to determine whether the ordering or administration of hospital treatment is influenced by time of day. Further, embodiments disclosed herein provide notifications and/or recommendations for providing treatment when it is most effective based on the same, thereby improving patient care and providing a practical application.

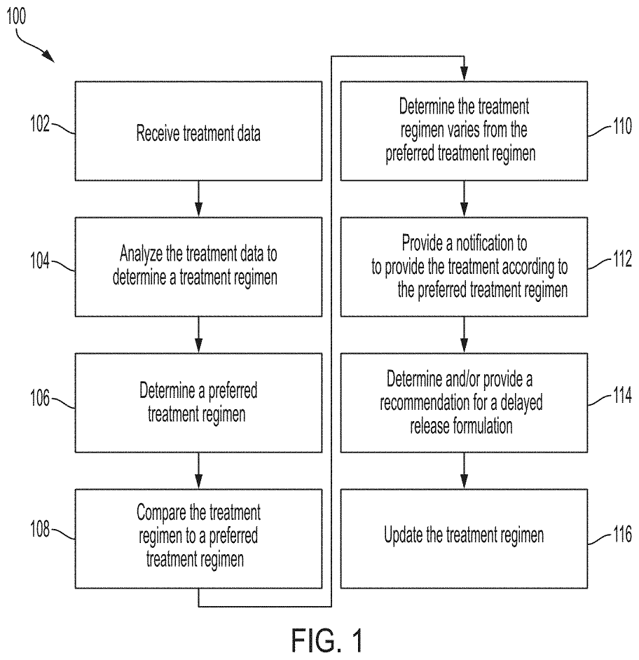

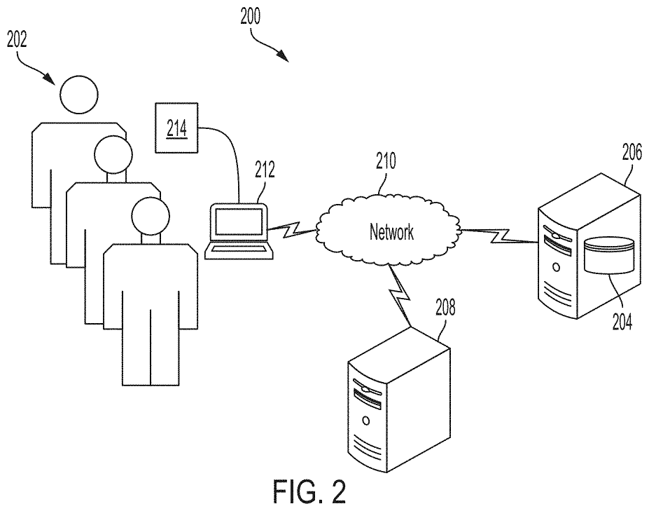

FIG. 1 illustrates a flow diagram of a method 100 for analyzing operational rhythms in hospital treatment and providing recommendations for hospital treatment based on the same. And, FIG. 2 is a block diagram of a system 200 that can be used to implement the method 100 of FIG. 1. These diagrams are merely examples, which should not unduly limit the scope of the claims. One of ordinary skill in the art would recognize many variations, alternatives, and modifications.

According to certain embodiments, the method 100 includes receiving treatment data for a treatment provided to a plurality of patients (block 102). The treatment can include, but is not limited to, administration of a therapeutic (e.g., drug) and/or a performance of a procedure (e.g., a surgery, a rehabilitation exercise, dialysis, a diagnostic test, cognitive behavioral therapy, etc.). The therapeutics may include any therapeutic administered at a hospital to a patient 202 including, for example, anti-hypertensives (e.g., hydralazine), anti-inflammatory drugs (e.g., methylprednisone), analgesics, anti-infectives, antihistamines, diuretics, vasodilators, beta-blockers, inotropes, pain therapeutics, and other drug classes commonly used in the hospital. In certain instances, the treatment data can include, but is not limited to, a type of therapeutic administered to a patient 202, a dosage of the therapeutic, a type of procedure performed on a patient 202, rhythmic data (e.g., a time of day that a therapeutic was administered to a patient 202, a time of day that a procedure was performed on a patient 202, a day of the week that a procedure is performed on a patient 202, a day of the week that a therapeutic was administered to a patient 202), and/or any other data pertaining to the treatment stored in an electronic health record (EHR).

In certain instances, the treatment data for a patient is entered into and included in the EHR for the patient. The EHRs may be stored on at least one database 204. The database 204 may be incorporated into and/or accessible via one or more healthcare software system servers 206, such as Epic Systems, Cerner, Meditech, CPSI, Medhost, Allscripts, etc.

In certain examples, an analytics server 208 receives the treatment data from database 204 and performs analytics on the treatment data, as explained in more detail below. In some examples, the analytics server 208 receives and/or retrieves the treatment data from the database 204 via an application programming interface (API) using, for example, a query language (e.g., SQL) and/or one or more standardized protocols for transfer of clinical and administrative data between software applications used by various healthcare providers (e.g., health level 7 (HL7)). Each EHR can have its own specific API and associated documentation. For example, documentation associate with Epic APIs can be found here: https://open.epic.com/Home/InteroperabilityGuide?whoAmI=developer&whatIWant=techSpecs, the contents of which is incorporated herein for all purposes. Additionally, or alternatively, the analytics performed on the treatment data can be performed by the server 206. In some embodiments, the analytics may be performed in real-time in order to identify potentially problematic events, for example, identifying and notifying a clinician of a significant dosage lag for a therapeutic prescribed to a patient 202.

According to certain embodiments, the server 206 and the analytics server 208 are communicatively coupled via a network 210. The network 210 may be, or include, any number of different types of communication networks such as, for example, a bus network, a short messaging service (SMS), a local area network (LAN), a wireless LAN (WLAN), a wide area network (WAN), the Internet, a P2P network, custom-designed communication or messaging protocols, and/or the like.

Referring to FIG. 1, the method 100 includes analyzing treatment data to determine a treatment regimen (block 104) and/or determining a preferred treatment regimen (block 106). Treatment data can include, but is not limited to, patient characteristics (e.g., sex, age, weight, medical history, and diagnosis), what type of therapeutic is ordered, how often a therapeutic is ordered and/or administered (i.e., dosing frequency), what dose of the therapeutic is ordered and/or administered, when a therapeutic is ordered (e.g., time of day and/or day of the week), when a therapeutic is administered (e.g., time of day and/or day of the week), the time between when a therapeutic is ordered from the pharmacy and when a therapeutic is available for administration (i.e., dose lag), the effectiveness of a therapeutic based on when it is administered (e.g., information about how the patients 202 respond to the treatment regimens), etc.

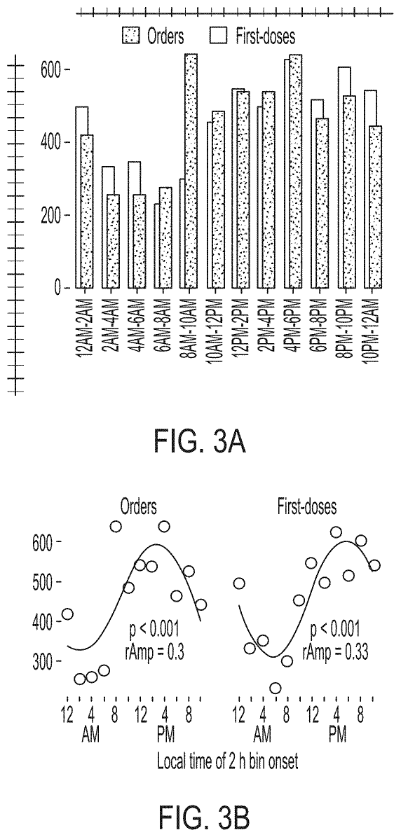

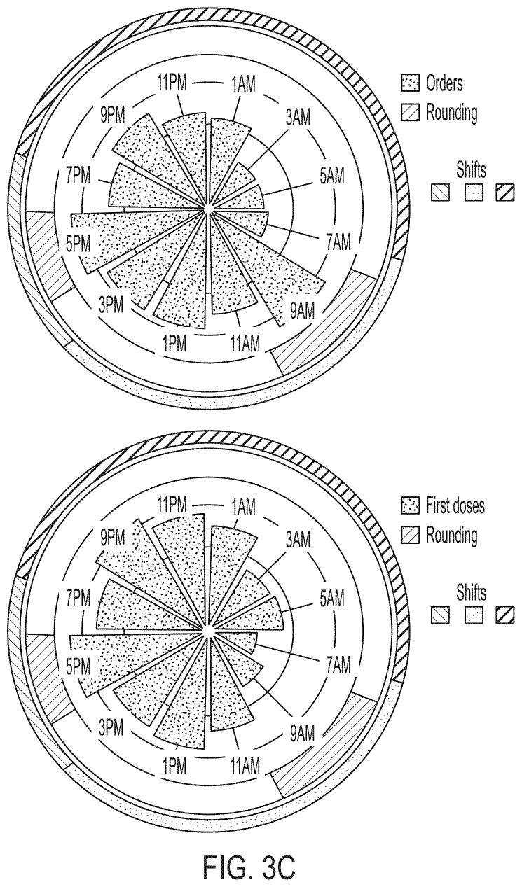

FIGS. 3A-6B are examples of analysis performed on treatment data in order to determine a treatment regimen and/or determine a preferred treatment regimen. The analysis included analyzing the treatment data of 12 therapeutics commonly administered to patients. Leveraging this large dataset of ˜500,000 doses, the embodiments examined the 24-hour distribution of order and administration times of multiple different drug classes, across different care units. The analysis found that treatment was strongly time-of-day dependent. Lastly, the embodiments explore the impact of dosing time on clinical outcomes, which is used to determine a preferred treatment regimen.

Referring to FIGS. 3A-3C, daily rhythms in the timing of hydralazine treatment are illustrated. Hydralazine is a vasodilator used to treat essential hypertension. The embodiments analyze the 24-hour distribution of order and administration times in 1,546 patients who received at least one dose of hydralazine, a commonly used antihypertensive in the ICU, at a tertiary pediatric hospital from 2010 to 2017. Characteristics of the patients (e.g., sex, age, and diagnosis) who received at least one dose of hydralazine are included in Table 1 below.

TABLE 1
Patient admissions and treatment summary

| | Age, y | | | | | |
|---|---|---|---|---|---|---|
| | 1-5 | 5-7 | 7-10 | 10-15 | 15-20 | >20 |
| Sex (%) | | | | | | |
| Female | 232 (40) | 239 (42) | 594 (39) | 685 (40) | 514 (42) | 1,088 (52) |
| Male | 349 (60) | 332 (58) | 936 (61) | 1,018 (60) | 699 (58) | 1,000 (48) |
| Diagnosis % | | | | | | |
| Fever | 7 | 10 | 11 | 12 | 6 | 5 |
| Respiratory distress | 3 | 2 | 3 | 3 | 2 | 3 |
| Hemophagocytic syndromes | 2 | 2 | 2 | 1 | 1 | 1 |
| Leukemia | 2 | 3 | 2 | 5 | 4 | 3 |
| Dehydration | 1 | 1 | 2 | 1 | 1 | 1 |
| Neutropenia | 1 | 2 | 2 | 2 | 2 | 1 |
| Renal failure/injury | 1 | 1 | 3 | 3 | 1 | 3 |
| Pneumonia | 1 | 1 | 2 | 2 | 2 | 2 |
| Cystic fibrosis | <1 | <1 | <1 | <1 | <1 | 3 |
| Other | 31 | 32 | 38 | 37 | 40 | 48 |
| Not reported | 51 | 44 | 34 | 35 | 40 | 29 |

Total admissions (n = 7,686) for 1,546 patients from 2010 to 2017.

In total, 5,485 orders for hydralazine were placed, each stipulating a dosing frequency of “once,” “as needed,” or “scheduled” (see Table 2 below). The embodiments track both order time (i.e., when a care provider places an electronic request for a drug) and dosing time (i.e., when the patient receives the drug) for each dose. Initial analyses considered only the first dose administered after each order.

TABLE 2
Treatment summary

| | | % order frequency | | | Doses | | % treatment unit | | |
|---|---|---|---|---|---|---|---|---|---|
| Drug | Orders | Once | Sched. | As needed | Total | % first | ICU | Floor | Other/NA |
| Hydralazine | 5,485 | 41 | 5 | 53 | 16,662 | 33 | 43 | 39 | 18 |
| Acetaminophen | 22,174 | 30 | 13 | 57 | 85,599 | 26 | 21 | 54 | 24 |
| Furosemide | 16,199 | 71 | 29 | 0 | 40,815 | 40 | 41 | 44 | 15 |
| Morphine | 13,084 | 28 | 7 | 64 | 65,463 | 20 | 46 | 33 | 21 |
| Lorazepam | 9,510 | 30 | 33 | 37 | 72,879 | 13 | 43 | 37 | 21 |
| Ondansetron | 8,005 | 20 | 37 | 44 | 62,061 | 13 | 14 | 61 | 25 |
| Fentanyl | 6,882 | 40 | 2 | 58 | 25,668 | 27 | 67 | 8 | 25 |
| Hydrocortisone | 6,623 | 26 | 67 | 7 | 45,996 | 14 | 24 | 58 | 18 |
| Diphenhydramine | 7,616 | 38 | 10 | 51 | 26,761 | 28 | 12 | 65 | 24 |
| Methylprednisolone | 3,818 | 32 | 67 | 1 | 20,118 | 19 | 35 | 44 | 21 |
| Labetalol | 2,441 | 33 | 38 | 29 | 17,021 | 14 | 41 | 37 | 22 |
| Vancomycin | 2,010 | 18 | 82 | 0 | 15,070 | 13 | 30 | 33 | 36 |

Treatment orders (n = 103, 847) and doses (n = 494, 113).

Hydralazine order and first-dosing times were nonuniformly distributed over 24 hours [Kuiper's test (3), P<0.01], marked by distinct morning-time surges and overnight lulls (see FIGS. 1A-1C). Nearly twice as many treatments were ordered between 8:00 AM and 6:00 PM (2,842) compared to 10:00 PM and 8:00 AM (1,652). The profiles were described by 24-hour rhythms using 3 separate Q:7 detection methods [cosinor analysis, described in G. Cornelissen, Cosinor-based rhythmometry. Theor. Biol. Med. Model. 11, 16 (2014), the entirety of which is incorporated herein by reference for all purposes, JTK_CYCLE described in M. E. Hughes, J. B. Hogenesch, K. Kornacker, JTK_CYCLE: An efficient nonparametric algorithm for detecting rhythmic components in genome-scale data sets. J. Biol. Rhythms 25, 372-380 (2010) and G. Wu, R. C. Anafi, M. E. Hughes, K. Kornacker, J. B. Hogenesch, MetaCycle: An integrated R package to evaluate periodicity in large scale data. Bioinformatics 32, 3351-3353 (2016), the entireties of which are incorporated herein by reference for all purposes, and RAIN described in P. F. Thaben, P. O. Westermark, Detecting rhythms in time series with RAIN. J. Biol. Rhythms 29, 391-400 (2014), the entirety of which is incorporated herein by reference for all purposes, P<0.05] (FIG. 3B). The morning surge in hydralazine order times coincides with team rounding and a medical staff shift change (FIG. 3C). Caretaker shifts at our institution include 7:00 AM to 7:00 PM, 7:00 AM to 3:00 PM, and 7:00 PM to 7:00 AM shifts. Team rounds typically occur from ˜7:30 AM to 10:30 AM and 4:00 PM to 6:00 PM. These schedules are common across the United States.

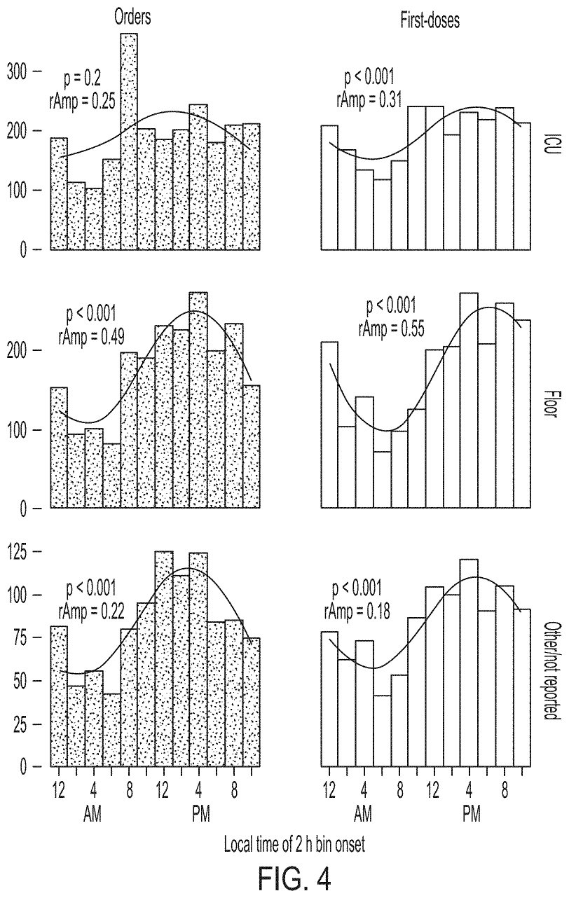

FIG. 4 illustrates rhythms in hydralazine orders and first doses across hospital units. Rhythms in hydralazine orders and first doses were consistent across patient age groups and treatment units—ICU or general floor. To determine whether the 24-h rhythm in dosing of hydralazine reflected immediate medical need for treatment, the embodiments analyze blood pressure (BP)s immediately prior to drug dosing (d-BP). For systolic d-BP, there were no statistically discernible differences between any 2-h time bins, in any age group (one-way ANOVA, P>0.05). For diastolic d-BP, although multivariate analysis detected marginally significant time-of-day differences in 2 of the 6 age groups (one-way ANOVA, P<0.05), post hoc analyses found Q:8 only one significant difference (Tukey's test, adjusted P<0.05) across all 2-h time bin comparisons. This suggests that 24-h rhythms in treatment were not solely driven by immediate medical need.

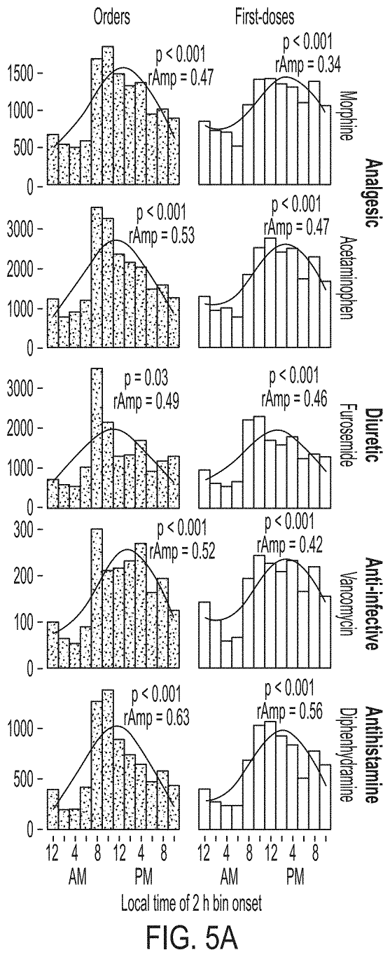

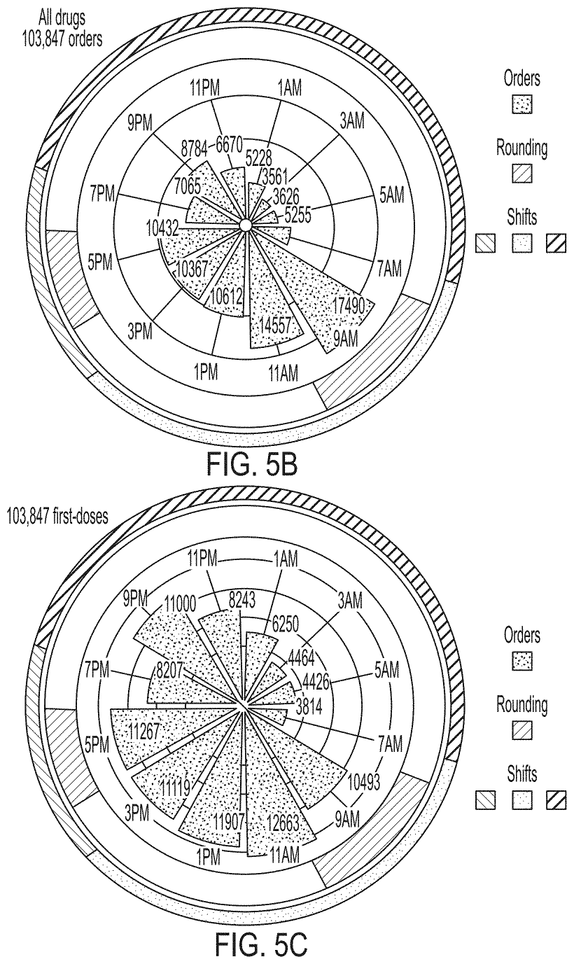

The embodiments next test whether the 24-h patterns in hydralazine use generalized to other therapies, including analgesics, anti-infectives, antihistamines, diuretics, and other drug classes commonly used in the hospital. FIGS. 5A-5C illustrate rhythms in treatment across drug classes coincide with hospital-wide operational activity. For each of the additional 11 drugs analyzed, order and first dose times were nonuniformly distributed (Kuiper's test, P<0.01), with characteristic morning surges and overnight lulls (see FIG. 5A). The majority (9 out of 11) were described by 24-h rhythms (cosinor, P<0.05; RAIN or JTK_CYCLE, P<0.05), regardless of treatment unit. No single model provided the best fit for all. Whereas cosinor methods fit well to profiles with smoother rises and falls, RAIN (an umbrella function) better fit several of the spiky (sawtooth) profiles. Overall, nearly one-third of the total 103,847 drug orders were placed during the 4-h time window from 8:00 AM to 12:00 PM. This morning surge in ordering first doses of all drugs coincides with team rounding (see FIGS. 5B and 5C).

The distribution of first doses resembled orders but was shifted by ˜1 hour to 2 hours (see FIGS. 5A-5C). At an example institution, orders are entered, filled by the central pharmacy, sent to the ordering site, and then administered by staff. This creates a time lag from order to first dose, which, interestingly, was also influenced by time of day. Lags were longest for orders placed from 8:00 AM to 12:00 PM (one-way ANOVA, P<0.05, 9 out of 12 drugs).

Previous analyses included all order types (“once,” “scheduled,” or “as needed”) but considered only the first doses from each order, which comprised 21% of all doses. Next, the embodiments compare first doses with “all other doses,” focusing on hydralazine as an example. As expected, first dose times were rhythmically distributed regardless of order type. Conversely, “all other doses” from scheduled orders had no discernible 24-hour rhythm. This was not surprising, as timings were stipulated (e.g., “take every 8 h”). Interestingly, “all other doses” from “as needed” orders (e.g., “take every 8 h as needed”) showed a weaker but still discernible 24-hour rhythm. Thus, although first doses showed the strongest 24-h patterns, decisions to administer subsequent doses may also be influenced by time of day. In sum, the analysis of the treatment data indicates the treatment regimen (i.e., first dose times) are primarily determined by order time+operational lag (block 104). While the embodiments discussed above discuss 12 types of therapeutics, the embodiments can be used to analyze the treatment data for any type of treatment in order to determine a treatment regimen.

Rhythms in hospital medicine may conflict with circadian biology, as dosing time influences responses to many types of treatment. For example, in the outpatient setting, short-acting antihypertensives may be most effective at lowering BP if taken before bedtime. Therefore, in certain embodiments, the method includes determining a preferred treatment regimen (block 106). In certain instances, determining a preferred treatment regimen includes, but is not limited to, an optimal time during the day for administering a therapeutic and/or performing a procedure, which can be patient specific based on responses to the treatment, examples of which are provided below.

Determining a preferred treatment regimen may include using statistical methods (e.g., linear or non-linear regression), machine learning, or both. For example, regarding machine learning, the method 100 may use the effectiveness of any of the treatment regimens as labeled data and train weights of an algorithm using supervised learning such that the output of the algorithm will be the treatment regimen that optimizes the effectives of a therapeutic included in the therapy regimen.

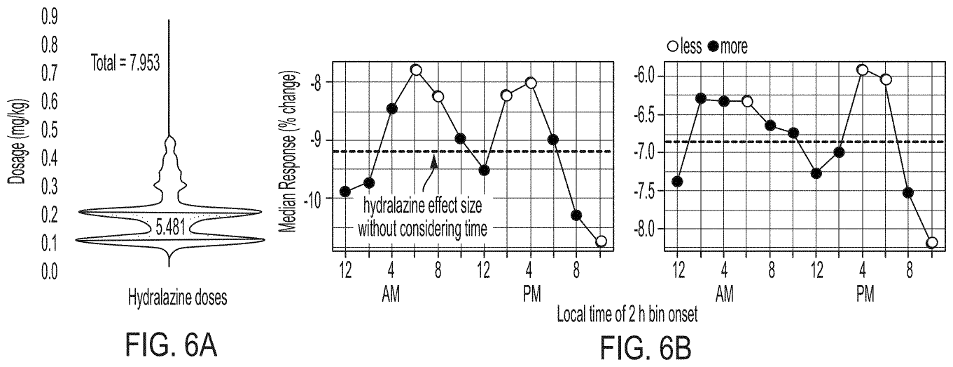

FIGS. 6A-6B illustrate clinical response to hydralazine varies by time of administration. In particular, FIGS. 6A-6B analyze inpatient responses to 7,953 doses of intravenous (IV) hydralazine as a function of dosing time. Response was computed as the percent change between dosing BP (just before dose) and mean BP over the 3 hours following each dose. To control for the impact of dosage, the embodiments stratify doses by concentration (milligrams per kilogram body weight) (see FIG. 6A).

The response to hydralazine varied over 24 hours (ANCOVA, diastolic P=2e−13; systolic P=6e−4) in the most common dosage group (0.1≥mg/kg≤0.2; n=5,481 of 7,953 doses) (see FIG. 6B). BP was more responsive to nighttime dosing (10:00 PM to 12:00 AM) compared to morning (6:00 AM to 10:00 AM) and late afternoon (2:00 PM to 6:00 PM) dosing (see FIG. 6B, Tukey's test, p-adjusted<0.05). Both diastolic and systolic responses followed this pattern, although differences in diastolic BP were stronger. In sum, nighttime hydralazine was associated with an ˜3 to 4% greater reduction in diastolic BP, on average, than morning dosing (Tukey's test, difference=−3.7, 95% CI upper level=−5.7, lower level=−1.1, adjusted P=1e−07). This effect was also independent of patient sex.

The embodiments did not detect robust 24-hour variation in response for any of the other 3 dosage groups, which included far fewer doses. For the majority of doses, the clinical response to hydralazine varied over 24 h. Order and first dosing times of antihypertensives, analgesics, anti-infectives, and other drug therapies all followed a 24-hour rhythm characterized by a morning surge and an overnight lull. There are no practice guidelines or institutional policies specifying time-of-day recommendations for any of the drugs in this study. The surge in orders (8:00 AM to 10:00 AM) coincides with rounding—when the medical care team visits each patient and collaboratively develops a treatment regimen. Orders for diagnostics, therapies, and referrals to specialty services are commonly placed during rounds or immediately thereafter, once final consensus is reached among the team.

Systemic bias toward treatment at a particular time of day is problematic. For example, anti-infectives should be administered when an infection is identified, whereas analgesics should be administered when there is pain. Neither of these happens selectively in the morning. In fact, pain may be more severe in the evening. Circadian clocks coordinate physiologic functions and may affect therapeutic responses according to the time of day. For example, patterns in BP may be closely tied to both endogenous circadian rhythms and the sleep-wake cycle. BP may decrease during sleep, often referred to as “dipping.” Cardiovascular disease may be more common in people that demonstrate non-dipping BPs while asleep. Circadian variability in absorption, metabolism, and excretion of drugs may be exploited to improve treatments for various disorders, including cardiovascular disease. Short-acting antihypertensives taken by millions of adults, for example, may be most effective before bedtime. Here, the embodiments show that this may also true in the pediatric hospital setting. The clinical response to an acute antihypertensive, hydralazine, varied by 3 to 4% over 24 hours. Paradoxically, patients were more responsive to therapy during the window of time (nighttime) when relatively few orders were placed and first doses administered.

Conversely, other treatments may be more effective if administered in the morning [e.g., vaccines, antipsychotics], or midday [e.g., corticosteroids, radiotherapy, cardiac surgery]. As such, the embodiments used above for determining a preferred treatment regimen for hydralazine can be used to determine a preferred treatment regimen (e.g., when a therapeutic should be administered) for any type of therapeutic (block 106).

Additionally, or alternatively, there may be no single optimal dosing time for all drugs and patients. Given the broad impact of circadian rhythms on physiology, the embodiments disclosed herein may determine the optimal time of day for therapeutic administration and/or performance of procedures for different patients based upon specific patient responses to administration of therapeutics and/or performance of procedures.

As stated above, hospital medical staff are available to provide treatment around the clock, and prevailing dogma is that treatment is given as needed regardless of time of day. However, the embodiments discussed above and below challenge this notion and reveal a potential barrier to best clinical care. Interestingly, more-frequent rounding by nurses may improve measures of patient care. Would medical rounding designed to be more evenly distributed over 24 hours improve clinical outcomes? Indeed, there are other long-standing aspects of hospital operation that have been challenged by the embodiments disclosed herein. For example, traditional continuous dim lighting in ICUs may lack a medical rationale, disrupts patients, and may impede recovery.

Because the administration of therapeutics and/or performance of procedures does not necessarily coincide with the time during which the therapeutic is most effective, the method 100 may include comparing the treatment regimen to a preferred treatment regimen (block 108) and determining whether the treatment regimen varies from the preferred treatment regimen (block 110). In instances where the treatment regimen varies from the preferred treatment regimen by a threshold, the method 100 may include providing a notification to administer the treatment based on the preferred treatment regimen (block 112). In at least some embodiments, a clinician can review the notification to administer the treatment according to the preferred treatment regimen and decide when to provide the treatment. Examples of thresholds include, but are not limited to, 15 minutes, 30 minutes, 1 hour, 2 hours, etc. In certain examples, the threshold varies depending on the type of therapeutic, the diagnosis of the patient, and/or the patient characteristics.

Additionally, or alternatively, the method 100 may include providing a notification to a user interface when a treatment is to be administered without comparing the treatment regimen to the preferred treatment regimen and/or determining the treatment regimen varies from the preferred treatment regimen (block 112).

In certain instances, an administration device 214 can deliver treatment. In these instances, the method 100 may include updating the treatment regimen to correspond to the preferred treatment regimen and administering the treatment via the administration device 214 to the patient 202.

In certain instances, the notification is provided to a clinician interface 212 (e.g., the interface for Epic Systems, Cerner, Meditech, CPSI, Medhost, Allscripts, and/or the like) (shown in FIG. 2). In certain embodiments, the notification may include the optimal time of day and/or be provided at the optimal time of day for administering the therapeutic and/or performing a procedure based on when the therapeutic and/or procedure will be most effective.

Additionally, or alternatively, the notification may include a time of day for when a therapeutic should be ordered depending on the dose lag for a therapeutic (i.e., the time between when a therapeutic is ordered from the pharmacy and when a therapeutic is available for administration) so that the therapeutic can be delivered during the optimal time of day. Additionally, or alternatively, the notification may include: an identification of therapeutics where long lags pose greatest potential health risks, prioritization of certain therapeutics based on health risk due to delay in administering a therapeutic, and/or suggested operational changes to speed pharmacy turnaround during known rush hours.

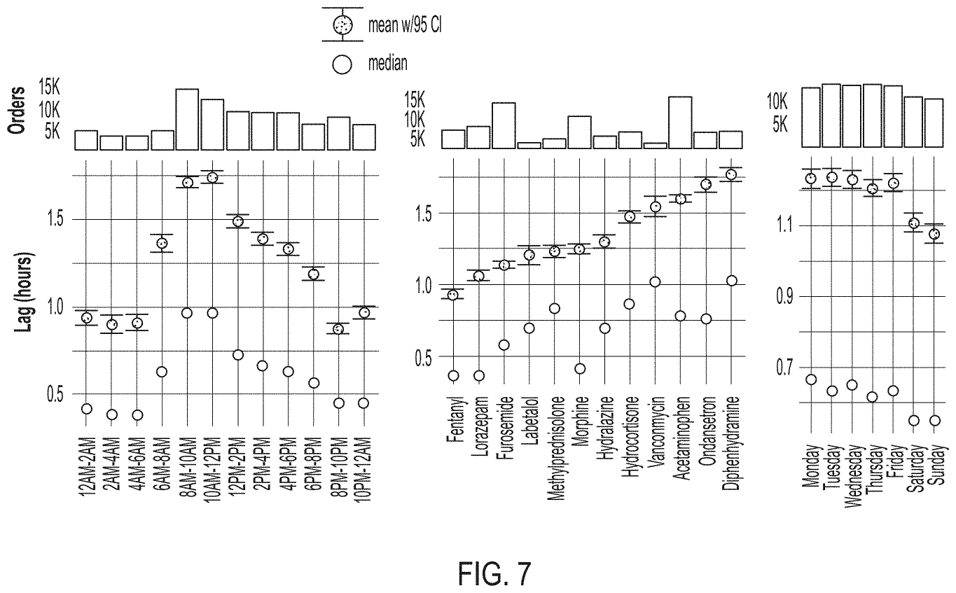

As an example, FIG. 7 illustrates the time between when a therapeutic is ordered and when it is administered (i.e., the dose lag) for approximately 100,000 orders. As illustrated, the dose lag is dependent upon the time of the order, the drug being ordered, and the weekday the drug is ordered. In certain instances, the dose lag may be due to variable capacity. For example, the longest dose lag coincides with times during which most therapeutics are ordered. Therefore, the notification to update the treatment regimen can include a recommended time for ordering a therapeutic based on the optimal time for delivering the therapeutic and the estimated dose lag for when the therapeutic is ordered and when the therapeutic is administered. In the example illustrated in FIG. 7, the dose lag is the longest for orders placed between 10 am-12 pm, so the notification can recommend ordering the therapeutic at an earlier time to account for the dose lag during these times.

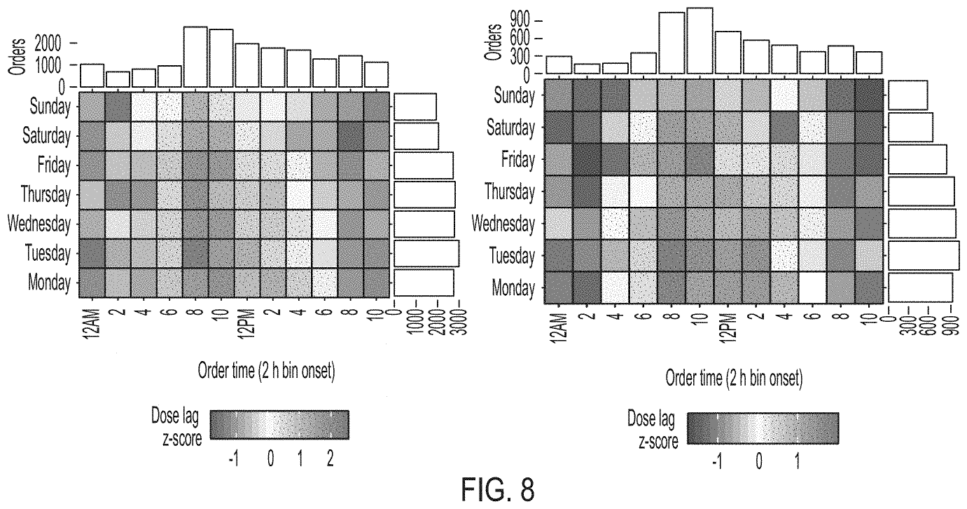

FIG. 8 illustrates the longest lag of the therapeutics illustrated in FIG. 7 are for acetaminophen and diphenhydramine. This lag may be due to the perceived medical urgency of the ordered therapeutic and the relative non-urgency of acetaminophen and diphenhydramine. Additionally, or alternatively, the lag may be due to the time to prepare the therapeutic, and/or the time to deliver the therapeutic as some therapeutics need to be physically walked to the point of administration. Similar to above, the notification to update the treatment regimen can include a recommended time for ordering a therapeutic based on the optimal time for delivering the therapeutic and the estimated dose lag for when the therapeutic is ordered and when the therapeutic is administered. In the example illustrated in FIG. 8, because the dose lag is longer for acetaminophen and diphenhydramine, the notification can recommend ordering acetaminophen and diphenhydramine at an earlier time to account for the dose lag for these therapeutics. Additionally, or alternatively, the preferred treatment regimen may be based on how long it takes to fill a prescription for the therapeutic. For example, if it takes a hospital two hours to fill a prescription, the preferred treatment regimen may order or may recommend ordering, via a notification, the prescription at least two hours prior to the time of day at which the therapeutic is most effective. Then, the therapeutic will be ready to be administered to the patient 202 at the time of day at which the therapeutic is most effective.

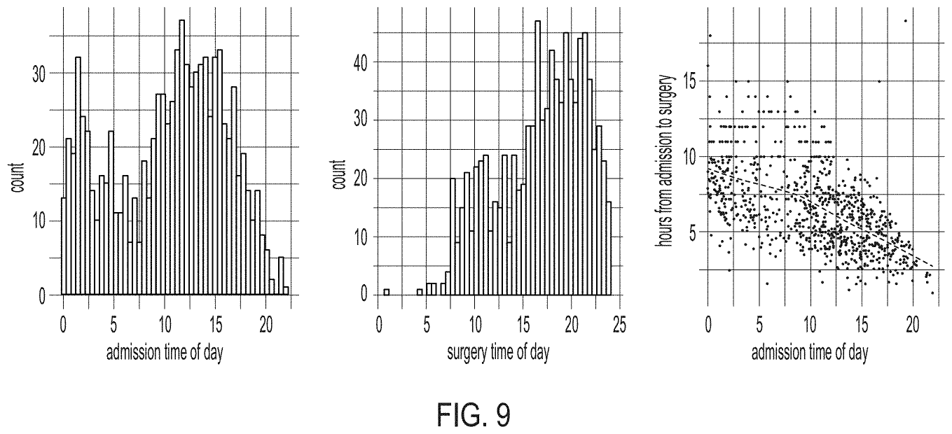

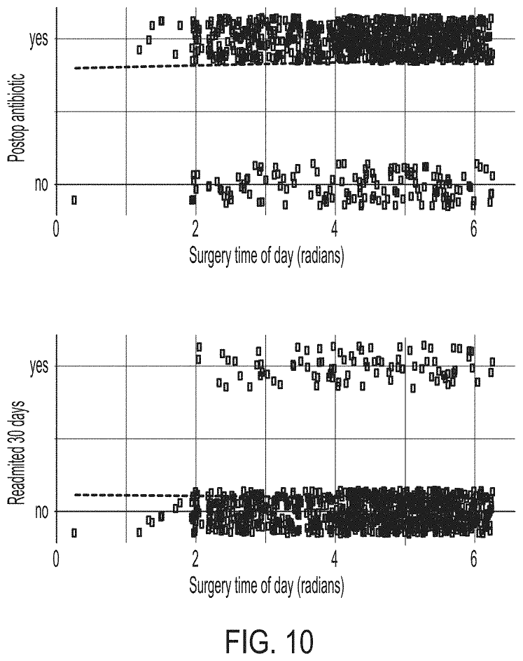

Additionally, or alternatively, providing a notification to provide the treatment according to the preferred treatment regimen (block 112), can be applied to when a procedure should be performed, in the event there is an optimal time for performing a procedure. As an example, FIG. 9 includes data for 906 appendectomies and illustrates the difference between when a patient is admitted for an appendicitis and when the surgery is performed (i.e., surgery lag) and FIG. 10 illustrates whether there is an association between time of surgery for an appendicitis and post-operation antibiotic use or readmission. In the illustrated embodiment, 762 of 906 patients received post-operative antibiotics and 88 of 906 patients were readmitted within 30 days. If there is a correlation between an increased surgery lag and increased post-operation antibiotic use or readmission, then the method 100 can include providing a notification to provide the treatment according to the preferred treatment regimen (block 112) for an appendicitis by, for example, decreasing the surgery lag. However, in the illustrated embodiment, there is no association between surgery lag and post-operative antibiotic use or readmission. As such, the method 100 may not include providing a notification for update the treatment regimen or indicate the current treatment regimen is adequate.

According to certain embodiments, the method 100 includes determining and/or providing a recommendation for a delayed release formulation of a therapeutic (block 114). For example, the method 100 may facilitate in the designing and/or administering of a delayed release formulation of the therapeutic to be used at home, at a clinic, or any location away from the clinic to optimally deliver the therapeutic when a time of dosing cannot be accurately controlled and/or if there is a standard time of day when a subject usually takes a medication (e.g., during the morning or at night). The time release mechanism controls the delay (i.e., how long) after the therapeutic is taken by a patient 202 for the therapeutic to reach its peak effectiveness. For example, if a therapeutic is most effective at around noon, but the standard time of day that a subject takes his/her medication is in the morning around 7 am, then the delayed release formulation of the therapeutic may be 5 hours so the subject can take his/her medicine at 7 am, but it will not reach its peak effectiveness until noon when the therapeutic is most effective. In at least some of these embodiments, the preferred treatment regimen may designate administration of the therapeutic at the same time of day (e.g., when a subject usually takes his/her medication), but will reach its peak effectiveness during different times of the day based upon when the therapeutic is most effective.

According to certain embodiments, the method 100 includes updating the treatment regimen (block 116). As set forth above, in at least some embodiments, a clinician can review the notification to update corresponding to the preferred treatment regimen and decide whether to update the treatment regimen. However, in certain embodiments, the treatment regimen can be automatically updated. Additionally, or alternatively, the method 100 may include providing a notification to an administration device 214 and the updated treatment regimen can be automatically administered via the administration device 214 to the patient 202, as stated above.

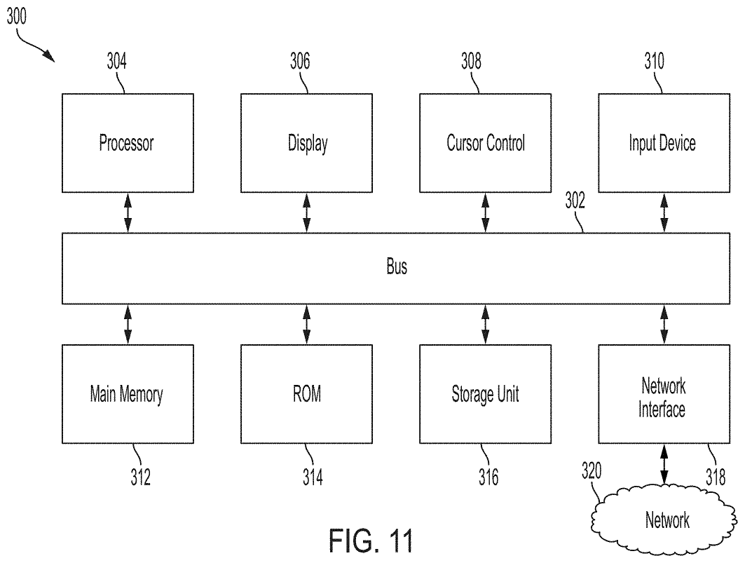

FIG. 11 is a block diagram of illustrative components of a computer system 300 for analyzing operational rhythms in hospital treatment and providing recommendations for hospital treatment based on the same. This diagram is merely an example, which should not unduly limit the scope of the claims. One of ordinary skill in the art would recognize many variations, alternatives, and modifications.

The computing system 300 includes a bus 302 or other communication mechanism for communicating information between, a processor 304, a display 306, a cursor control component 308, an input device 310, a main memory 312, a read only memory (ROM) 314, a storage unit 316, and/or a network interface 318. In some embodiments, the bus 302 is coupled to the processor 304, the display 306, the cursor control component 308, the input device 310, the main memory 312, the read only memory (ROM) 314, the storage unit 316, and/or the network interface 318. And, in certain embodiments, the network interface 318 is coupled to a network 320 (e.g., the network 210).

In some embodiments, the processor 304 includes one or more general purpose microprocessors. In some embodiments, the main memory 312 (e.g., random access memory (RAM), cache and/or other dynamic storage devices) is configured to store information and instructions to be executed by the processor 304. In certain embodiments, the main memory 312 is configured to store temporary variables or other intermediate information during execution of instructions to be executed by processor 304. For embodiment, the instructions, when stored in the storage unit 316 accessible to processor 304, render the computing system 300 into a special-purpose machine that is customized to perform the operations specified in the instructions (e.g., the method 100). In some embodiments, the ROM 314 is configured to store static information and instructions for the processor 304. In certain embodiments, the storage unit 316 (e.g., a magnetic disk, optical disk, or flash drive) is configured to store information and instructions.

In some embodiments, the display 306 (e.g., a cathode ray tube (CRT), an LCD display, or a touch screen) is configured to display information to a user of the computing system 300. In some embodiments, the input device 310 (e.g., alphanumeric and other keys) is configured to communicate information and commands to the processor 304. For embodiment, the cursor control 308 (e.g., a mouse, a trackball, or cursor direction keys) is configured to communicate additional information and commands (e.g., to control cursor movements on the display 306) to the processor 304.

Although the invention has been described in detail with reference to certain preferred embodiments, variations and modifications exist within the spirit and scope of the invention as described and defined in the following claims.

What is claimed is:

1. A server configured to analyze operational rhythms in hospital treatment and provide recommendations based on the same, the server comprising:
one or more processors; and
memory comprising instructions that, when executed, cause the one or more processors to:
receive, from a database, treatment data corresponding to a treatment provided to a plurality of patients;
determine a preferred treatment regimen based upon the treatment data, wherein the preferred treatment regimen comprises a time of day the treatment is more effective than other times of the day based upon responses to the treatment; and
provide a notification to a user interface to provide the treatment at the time of the day the treatment is more effective.

2. The server of claim 1, wherein the treatment corresponds to administration of a therapeutic, a performance of a procedure, or a combination thereof.

3. The server of claim 2, wherein the procedure is a surgery, dialysis, a diagnostic test, cognitive behavioral therapy, or a combination thereof.

4. The server of claim 2, wherein the treatment is at least one therapeutic selected from the following group of therapeutics: anti-hypertensives, anti-inflammatories, analgesics, anti-infectives, antihistamines, diuretics, vasodilators, beta-blockers, inotropes, pain therapeutics, or a combination thereof.

5. The server of claim 1, wherein to determine the preferred treatment regimen, the memory comprises instructions that, when executed, cause the one or more processors to use statistical methods, machine learning, or a combination thereof to determine what time of day the response to the treatment is more effective than other times of the day.

6. The server of claim 5, wherein the statistical methods comprise cosinor regression.

7. The server of claim 2, the memory comprising instructions that, when executed, cause the one or more processors to determine a delayed release formulation that corresponds to a difference between (i) a time of day when the therapeutic is more effective than other times of the day and (ii) a standard time of day a patient takes the therapeutic when the patient is at a location other than a patient care facility.

8. The server of claim 7, wherein the standard time of day is in the morning.

9. The server of claim 8, wherein the standard time of day is between 5 am and 9 am.

10. The server of claim 7, wherein the standard time of day is in the evening.

11. The server of claim 8, wherein the standard time of day is between 8 pm and 12 am.

12. The server of claim 1, wherein the treatment regimen comprises a time of day the treatment is provided.

13. The server of claim 1, wherein the notification includes an anticipated dose lag for a therapeutic.

14. The server of claim 13, wherein the anticipated dose lag is based on at least one hospital parameter.

15. A method for analyzing operational rhythms in hospital treatment providing recommendations based on the same, the method comprising:
receive, from a database, treatment data corresponding to a treatment provided to a plurality of patients;
determine a preferred treatment regimen based upon the treatment data, wherein the preferred treatment regimen comprises a time of day the treatment is more effective than other times of the day based upon responses to the treatment; and
provide a notification to a user interface to provide the treatment at the time of the day the treatment is more effective.

16. The method of claim 15, wherein the treatment corresponds to administration of a therapeutic, a performance of a procedure, or a combination thereof.

17. The method of claim 16, wherein the procedure is a surgery, dialysis, cognitive behavioral therapy, a diagnostic test, or a combination thereof.

18. The method of claim 16, wherein the treatment is at least one therapeutic selected from the following group of therapeutics: anti-hypertensives, anti-inflammatories, analgesics, anti-infectives, antihistamines, diuretics, vasodilators, beta-blockers, inotropes, pain therapeutics, or a combination thereof.

19. The method of claim 15, wherein determining the preferred treatment regimen comprises using statistical methods, machine learning, or a combination thereof to determine what time of day the response to the treatment is more effective than other times of the day.

20. The method of claim 19, wherein the statistical methods comprise cosinor regression.

21. The method of claim 15, further comprising determining a delayed release formulation that corresponds to a difference between (i) a time of day when the therapeutic is more effective than other times of the day and (ii) a standard time of day a patient takes the therapeutic when the patient is at a location other than a patient care facility.

22. The method of claim 21, wherein the standard time of day is in the morning.

23. The method of claim 22, wherein the standard time of day is between 5 am and 9 am.

24. The method of claim 21, wherein the standard time of day is in the evening.

25. The method of claim 24, wherein the standard time of day is between 8 pm and 12 am.

26. The method of claim 15, wherein the treatment regimen comprises a time of day the treatment is provided.

27. The method of claim 15, wherein the notification includes an anticipated dose lag for a therapeutic.

28. The method of claim 27, wherein the anticipated dose lag is based on at least one hospital parameter.
